(19) United States
(12) Patent Application Publication
(10) Pub. No.: US 2019/0247090 A1
(43) Pub. Date: Aug. 15, 2019

(54) MODIFIED VERESS NEEDLE FOR USE IN PERITONEAL DIALYSIS CATHETER INSERTION

(71) Applicant: University of Manitoba, Winnipeg (CA)

(72) Inventor: Sean Armstrong, Winnipeg (CA)

(21) Appl. No.: 16/272,460

(22) Filed: Feb. 11, 2019

Related U.S. Application Data
(60) Provisional application No. 62/629,455, filed on Feb. 12, 2018.

(51) Int. Cl. A61B 17/34; A61M 25/01; A61M 25/09

(52) U.S. Cl. CPC A61B 17/3474; A61M 5/158; A61M 25/09041; A61M 25/0102

(57) ABSTRACT

A Veress needle assembly for a peritoneal dialysis catheter guide wire has (i) a hollow needle with a pointed end, (ii) an inner tube received within the hollow needle so as to be slidable relative to the hollow needle in the longitudinal direction between a first position in which the first end of the inner tube protrudes longitudinally beyond the pointed end of the hollow needle and a second position in which the first end of the inner tube is retracted so that the pointed end is unobstructed by the inner tube, and (iii) a biasing mechanism to bias the inner tube towards the first position. The inner tube includes a fluid passage having an outlet opening at the first end of the inner tube that is oriented to allow the peritoneal dialysis catheter guide wire to readily the outlet opening when inserted through the fluid passage of the inner tube.

This application claims the benefit under 35 U.S.C. 119(e) of U.S. provisional application Ser. No. 62/629,455, filed Feb. 12, 2018.

### FIELD OF THE INVENTION

The present invention relates to a method of guiding insertion of a peritoneal dialysis catheter guide wire, and more particularly the present invention relates to a modified Veress needle assembly which is used in placing the guide wire that subsequently guides insert of the peritoneal dialysis catheter.

### BACKGROUND

The percutaneous technique of placing the peritoneal dialysis catheter is a widely used technique. One method involves the use of a conventional Veress needle assembly for access to the peritoneal cavity. The Veress needle has a double point: a cutting point formed by an outer hollow needle, and an inner member within the outer hollow needle that defines a retractable blunted point which is atraumatic. When the needle is pressed to the abdominal wall, the blunted point is retracted by tissues with high resistance, for example muscle, and the cutting point assists penetration; however, organs with low resistance, for example the bowel, do not retract the retractable point. Once the peritoneal cavity has been accessed, the Veress needle assembly then requires disassembly to remove the retractable inner member to allow passage of a guide wire into the peritoneal cavity which then subsequently guides insertion of the peritoneal dialysis catheter. Although a fluid passage is provided through the inner member in a typical Veress needle assembly, the fluid passage is intentionally designed to exit the retractable blunted point in a shielded location within the cylindrical side wall surface of the inner member and is thus unsuitable for passage of a catheter guide wire therethrough.

### SUMMARY OF THE INVENTION

According to one aspect of the invention there is provided a method of guiding insertion of a peritoneal dialysis catheter guide wire, the method comprising:

providing a needle assembly comprising: (i) a hollow needle having a hollow passage therein extending in a longitudinal direction between a first end of the hollow needle which is pointed and a second end of the hollow needle opposing the first end, (ii) an inner tube received within the hollow passage of the hollow needle so as to be slidable relative to the hollow needle in the longitudinal direction in which the inner tube is elongate in the longitudinal direction of the hollow needle between a first end of the inner tube which is in proximity to the first end of the hollow needle and a second end of the inner tube which is proximity to the second end of the hollow needle, and in which the inner tube is longitudinally slidable relative to the hollow needle between a first position in which the first end of the inner tube protrudes longitudinally beyond the pointed first end of the hollow needle and a second position in which the first end of the inner tube is retracted into the hollow needle relative to the first position so that the pointed first end of the hollow needle is unobstructed by the first end of the inner tube, (iii) a biasing mechanism operatively coupled between the inner tube and the hollow needle at the second end of the hollow needle so as to bias the inner tube from the second position towards the first position, and (iv) a fluid passage extending longitudinally through the inner tube from the second end of the inlet tube to an outlet opening at the first end of the inner tube for passing the intravenous fluid therethrough;

and inserting the peritoneal dialysis catheter guide wire into the fluid passage of the inner tube through the second end of the inlet tube so that the guide wire exits the outlet opening of inner tube.

According to a second aspect of the present invention there is provided a Veress needle assembly for use with a peritoneal dialysis catheter guide wire, the assembly comprising:

a hollow needle having a hollow passage therein extending in a longitudinal direction between a first end of the hollow needle which is pointed and a second end of the hollow needle opposing the first end;

an inner tube received within the hollow passage of the hollow needle so as to be slidable relative to the hollow needle in the longitudinal direction, the inner tube being elongate in the longitudinal direction of the hollow needle between a first end of the inner tube which is in proximity to the first end of the hollow needle and a second end of the inner tube which is proximity to the second end of the hollow needle;

the inner tube being longitudinally slidable relative to the hollow needle between a first position in which the first end of the inner tube protrudes longitudinally beyond the pointed first end of the hollow needle and a second position in which the first end of the inner tube is retracted into the hollow needle relative to the first position so that the pointed first end of the hollow needle is unobstructed by the first end of the inner tube;

a biasing mechanism operatively coupled between the inner tube and the hollow needle at the second end of the hollow needle so as to bias the inner tube from the second position towards the first position;

a fluid coupler operatively connected to the second end of the inner tube for connection to a source of intravenous fluid;

the inner tube including a fluid passage extending longitudinally therethrough from the fluid coupler at the second end of the inlet tube to an outlet opening at the first end of the inner tube for passing the intravenous fluid therethrough;

the outlet opening of the fluid passage at the first end of the inner tube being oriented relative to the fluid passage so as to allow the peritoneal dialysis catheter guide wire to readily pass through the fluid passage and exit the outlet opening when inserted into the fluid passage through the second end of the inner tube.

The outlet opening is preferably located in an end wall at the first end of the inner tube, concentrically with the fluid passage in the inner tube. The outlet opening is preferably reduced in diameter relative to an outer diameter of the inner tube so as to define an annular rim portion about the outlet opening in an end wall of the inner tube at the first end thereof. Preferably, the outer surface of the annular rim portion is generally convex.

The modified needle can be used for similar procedures as the original needle such as insufflation but would also allow for direct passage of a wire through its tip without requiring dismantling. This would allow for maintaining the protection of the dull retractable tip during any Seldinger technique (Seldinger technique. A generic eponymous term for any guide wire procedure, named after Swedish radiologist Dr Sven-Ivar Seldinger (1921-1998), who introduced the procedure in 1953, which allows safe access to blood vessels and hollow organs.)

As noted above, a typical Veress needle has a fluid passage with an outlet hole on the side and does not allow for a guide wire to be passed through.

The hole in the end of the catheter has also be designed to be large enough that it allows for the same rate of fluid flow as the original.

Both reusable stainless steel needles as well as disposable ones may be contemplated within the scope of the present invention.

The modified Veress needle may also be offered as a complete kit with wire, Veress needle and catheter. Video and online access for questions/video chapters would also be available.

### BRIEF DESCRIPTION OF THE DRAWINGS

One embodiment of the invention will now be described in conjunction with the accompanying drawings in which:

FIG. 1 is a side elevational view of the Veress needle assembly according to the present invention prior to attachment of a fluid coupler;

FIG. 2 is a side elevational view of the needle assembly with a fluid coupler supported thereon;

FIG. 3 is an exploded elevational view of the needle assembly according to FIG. 1;

FIG. 4 is a longitudinal cross-sectional view of the housing at one end of the needle assembly in a first protruding position of the needle assembly;

FIG. 5 is a longitudinal cross-sectional view of the housing at one end of the needle assembly in a second retracted position of the needle assembly; and

FIGS. 6 through 12 are schematic representations of a series of steps involving use of the needle assembly according to FIG. 1 for placement of a peritoneal dialysis catheter guide wire.

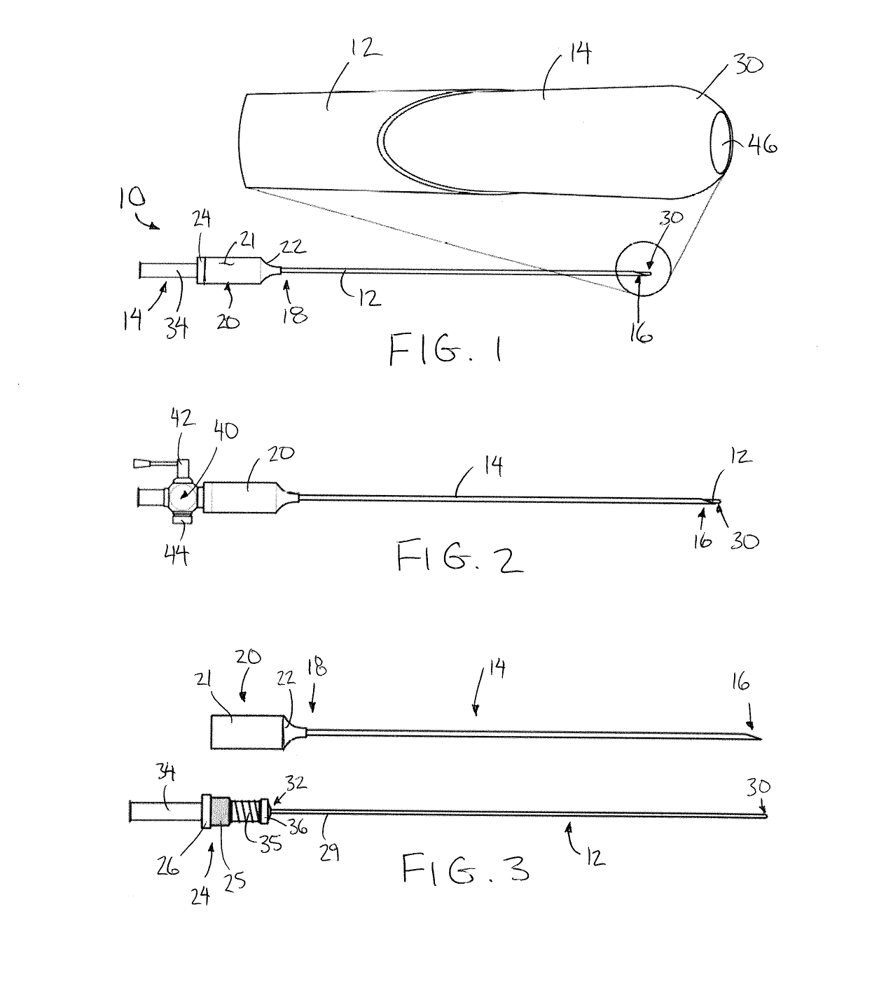

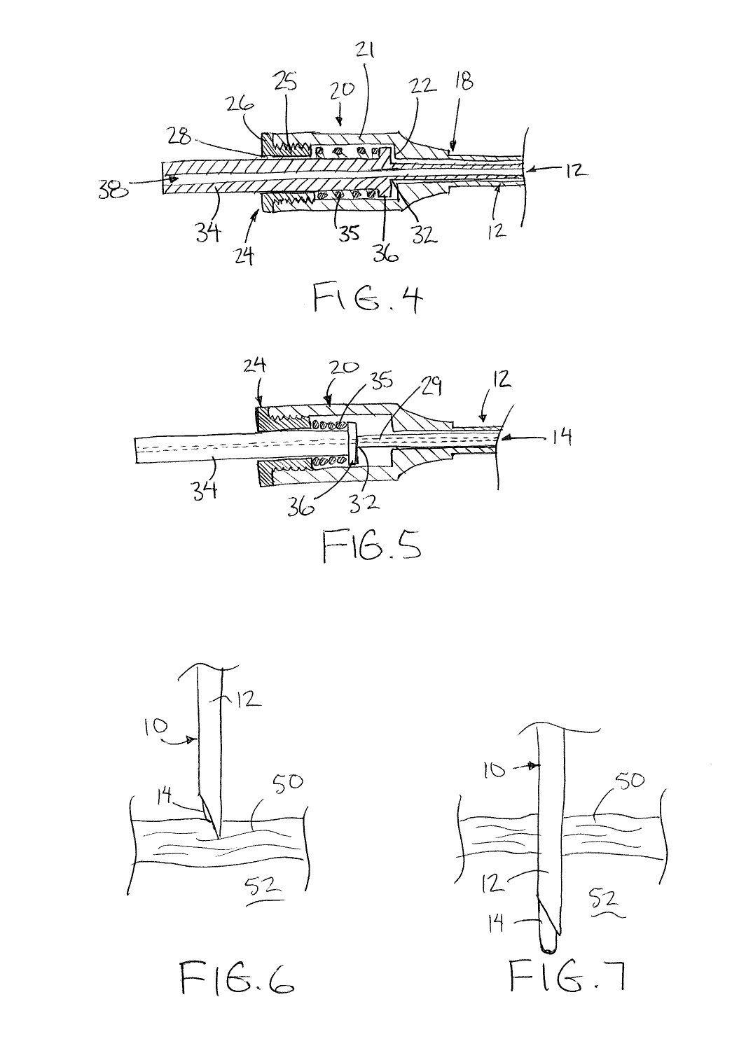

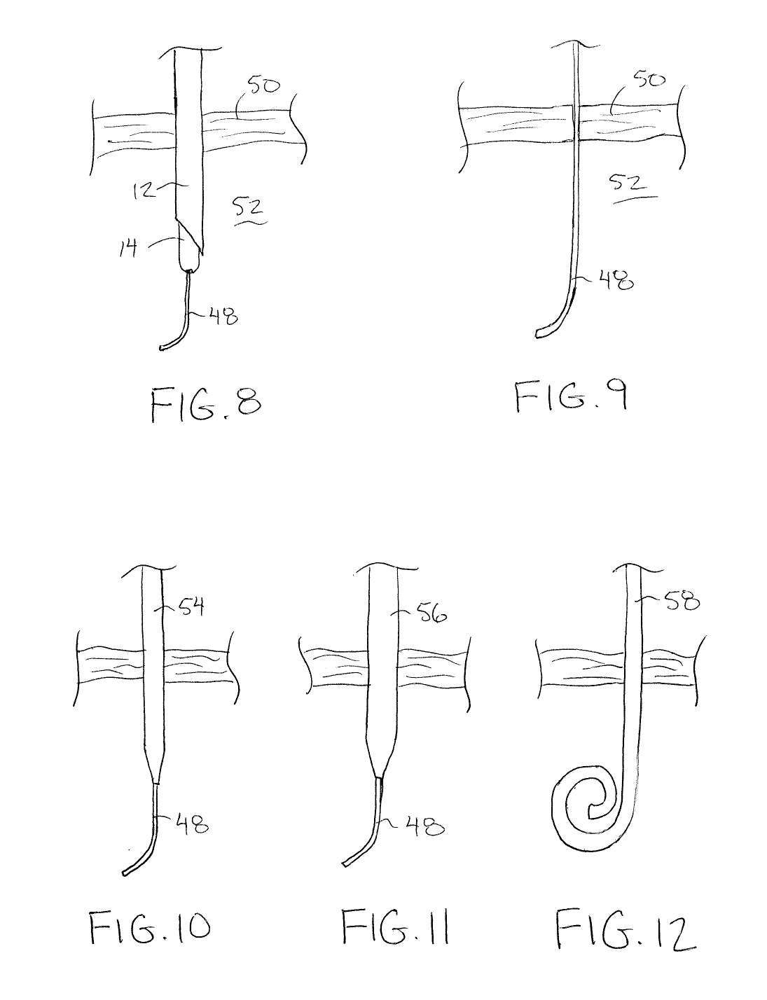

In the drawings like characters of reference indicate corresponding parts in the different figures.

### DETAILED DESCRIPTION

Referring to the accompanying figures there is illustrated a modified Veress needle assembly generally indicated by reference numeral 10. The Veress needle assembly 10 is particularly suited for use in peritoneal dialysis catheter insertion as described in the following.

The needle assembly generally includes a hollow needle 12 which receives an inner tube 14 therein so as to be longitudinally slidable relative to the hollow needle.

The hollow needle 12 is typically a rigid, metal needle which is elongate in a longitudinal direction from a first end 16 which is sharpened to form a pointed tip to a second end 18 opposite the first end. An elongate hollow passage spans the full length of the needle between the first end and second end thereof. A housing 20 is fixed onto the second end of the hollow needle 12. The housing 20 comprises a generally cylindrical side wall defining a hollow cavity therein which is enlarged in diameter relative to the hollow needle 12. An inner end wall 22 and the annular gap between the cylindrical side wall 21 of the housing and the reduced diameter of the hollow needle 12 at the junction of the second end of the needle 12 and the inner end of the housing 20. A central opening is provided in the inner end wall 12 which is equal in diameter to the diameter of the hollow passage so that the cavity within the housing 20 is in open communication with the passage within the hollow needle.

The outer end of the side wall 21 of the housing is internally threaded for receiving an end cap 24 therein. The end cap 24 is a collar having an inner portion 25 which is generally cylindrical and externally threaded for being received in mating connection within the outer end of the housing 20, and an outer portion 26 which is increased in diameter relative to the inner portion at the outer end of the cap for abutment with an outer end of the cylindrical side wall 21 of the housing when the end cap is threaded into the housing. A central opening 28 is provided within the end cap to communicate longitudinally therethrough.

The inner tube 14 includes (i) a needle portion 29 which is rigid and elongate in a longitudinal direction between a first end 30 for being positioned in proximity to the first end 16 of the hollow needle to a second end 32 for being positioned in proximity to the second end 18 of the hollow needle, and (ii) an end portion 34 which is mounted at the second end of the needle portion 29 and which is increased in outer diameter relative to the needle portion. More particularly, the needle portion 29 has an outer diameter which closely fits within the inner diameter of the hollow passage within the hollow needle 12 to be received longitudinally slidably therein, while the end portion 34 has an outer diameter which closely correspond to the inner diameter of the opening 28 extending through the end cap 24 of the housing but which is slightly reduced in diameter relative to the inner diameter of the main cavity of the housing 20. In this manner, the end portion 34 is longitudinally slidable within the housing while protruding longitudinally outward beyond the outer end of the housing by extending through the end caps 24. The needle portion 29 is longer in the longitudinal direction in its length between the first and 30 thereof and the junction with the end portion 34 as compared to the length of the needle portion between the first end 16 thereof and the junction of the second end 18 of the needle at the housing 20. In this manner, when the end portion 34 of the inner tube is abutted against to the inner end wall 22 of the housing, the first end of the inner tube 14 protrudes beyond the first end of the hollow needle 12 so that a first end portion of the inner tube 14 shields the pointed tip of the hollow needle by positioning apex of the pointed tip in close proximity alongside the body of the inner tube 14.

The annular gap between the outer diameter of the end portion 34 of the inner tube 14 and the interior diameter of the housing 20 receives a helical spring therein. An annular collar 36 is fixed at the inner end of the end portion 34 in which the outer diameter of the annular collar is increased relative to the remainder of the end portion 34 while remaining receivable within the interior diameter of the housing so that the collar 36 is longitudinally slidable with the inner tube relative to the surrounding housing. The helical spring 35 is received under compression between the annular collar 36 and the inner end of the end cap 34 to create a biasing force which urges the inner tube 14 into a first position relative to the hollow needle in which the first end of the inner tube protrudes beyond and shields the pointed tip at the first end 16 of the hollow needle. When pushing the needle assembly 10 through dense tissue so that the tissue imposes a resistance force on the first end of the inner tube 14 which exceeds the biasing force of the spring, the inner tube 14 will retract to a second position in which the first end of the inner tube 14 is retracted longitudinally inward relative to the pointed tip at the first end 16 of the hollow needle to expose the pointed tip for ready penetration through the dense tissue. Once the first end of the hollow needle has passed through the denser tissue, the biasing force of the spring can return the inner tube 14 to the first position.

The inner tube 14 includes a fluid passage 38 extending longitudinally therethrough across a full length of both the needle portion 29 and the end portion 34 of the inner tube. A fluid coupler 40 is mounted at the outer end of the end portion 34 which protrudes outwardly beyond the housing 20 through the full range of movement of the inner tube between the first and second positions thereof so that the fluid coupler does not interfere with the movement of the inner tube between the two positions. The fluid coupler includes a valve member 42 and an inlet coupling 44 for coupling to a fluid source, for example a supply line from a source of intravenous fluid.

At the first end 30 of the inner tube 14 the outermost end wall of the body of the inner tube is a rounded tip which is generally semicircular in shape. A central opening 46 is provided in the semicircular end wall at a central location which is concentric with the fluid passage 38, having diameter which is approximately equal to the inner diameter of the fluid passage 38 so as to be in alignment therewith. The central opening 46 is reduced in diameter relative to the outer diameter of the inner tube at the first end thereof so that an annular rim is defined about the central opening which is generally convex for ease in guiding the needle assembly through tissues within the body of a patient. The interior diameter of the fluid passage 38 and the central opening 46 are suitably sized to allow passage of a guide wire 48 therethrough in which the guide wire is of the type used for peritoneal dialysis catheter insertion.

In further embodiments, the outlet opening 46 at the first end of the body may be positioned in a non-concentric manner relative to the fluid passage, or even in a side wall of the inner tube 14, but in each instance shape of the fluid passage communicating with the outlet opening 46 is devoid of any sharp corners or sharp angle bends which would prevent ready passage of a guide wire inserted longitudinally through the fluid passage 38 from the outer end of the end portion 34 of the inner tube to the outlet opening 46 at the first end of the inner tube.

When using the Veress needle assembly 10 in the insertion of a peritoneal dialysis catheter guide wire, the fluid coupler 40 is initially coupled to a source of intravenous fluid which fills the fluid passage 38 so that fluid can pass out of the outlet opening 46 at the first end of the inner tube 14. The needle assembly 10 is then penetrated initially through a first layer of dense tissue as shown in FIGS. 6 and 7 in which the resistive force of the dense tissue causes the inner tube 14 to be retracted from the first position to the second position thereof allowing the pointed tip at the first end 16 of the hollow needle to penetrate through the tissue layer. The biasing force of the spring then returns the inner tube 14 into the first position shown in FIG. 7 after passing through the denser tissue. Continued application of an intravenous fluid through the fluid coupler 40 allows a peritoneal cavity 52 within the body of the patient to be partly filled with the intravenous fluid. The source of the intravenous fluid can then be disconnected from the needle assembly 10 while the inner tube 14 remains in the first position protecting the pointed tip at the first end 16 of the hollow needle 12. The guide wire 48 is then inserted longitudinally through the fluid passage 38 to exit the outlet opening 46 into the cavity 52. The guide wire 48 can then be left in place as the needle assembly 10 is removed. Subsequent steps involve placing a first small diameter dilator 54 over the guide wire to enlarge the opening through the dense tissue layers 50. The first dilator 54 is then removed and then a second dilator 56 of increased outer diameter is inserted over the same guide wire 48 which remains in place. Upon removal of the second dilator 56, the peritoneal dialysis catheter 58 can then be inserted over the guide wire to allow the guide wire 48 to be subsequently withdrawn from the patient.

An overview of the overall procedure for peritoneal dialysis catheter insertion will now be described in further detail. The patient is assessed in regards to hernias, excess skin and areas of infection to avoid. Level of pant/belt line are marked out for exit site planning of catheter as well as to which side the patient sleeps on so as to best identify which side of the body the catheter should be placed. Using the superior aspect of the pubic symphysis as the point of reference for catheter placement the deep and superficial cuff/exit site are then marked out.

Once the outline of the catheter has been made the patient's abdomen is sterilized with chlorhexidine and then draped. The outlined catheter points of reference including the planned plane to the rectus muscle then receive local anesthetic usually 40 to 50 cc of 1% lidocaine with epinephrine. An horizontal incision (3 to 4 cm) is then made using a 11 gauge scalpel over the outlined point of the deep cuff in reference to the superior aspect of the pubic symphysis. A Kelley's forceps is used to dissect down to the anterior rectus sheath and further local anesthetic is given into the rectus muscle to the point of the posterior rectus sheath and peritoneum.

A modified Veress needle is the most important instrument in this next step. As noted above, the Veress needle is a spring loaded instrument that has a sharp outer component with a safety inner dull point which springs out when entering an area of less resistance such as the peritoneal space. The Veress needle which is attached to an extension IV line to a saline drip is then advanced through this incision at a 30 to 45 degree angle. As the Veress needle is being advanced the patient is being instructed to “pop up” their abdomen to give counter traction. As the Veress needle enters the peritoneal space the Veress needle's protective dulled inner spring loaded point is advanced/released which is designed to reduce possible trauma. The physician should be able to appreciate both a tactile sensation as well as an auditory click as the inner dulled point is released. Further confirming that the Veress needle has entered the peritoneal space is that the saline chamber which is connected to the Veress needle will show a full flow versus slow drip when in an unobstructed space. The peritoneal space is then instilled with approximately 1.5 to 2 litres of dialysate. This step is made to expand the space to reduce the risk of bowel perforation and reduce resistance to wire/catheter advancement.

Once the peritoneal space has been filled, a guide wire (depending on centre choice and accessibility) is placed through. The guide wire may be a flexible stainless steel wire or a very stiff Amplatz wire.

When using a traditional Veress needle according to prior art procedures, the first stage or inner tube of the Veress needle is removed from the hollow needle to allow for the passage of the guide wire through the hollow needle. In this instance concern is made due to the fact that the second stage or sharp point remains in the abdomen unprotected for a short period of time.

Contrary to the prior art, the modified Veress needle according to the present invention does not require dismantling as the opening of the needle is at the end and not the side. Accordingly, the sharp pointed tip of the hollow needle remains shielded by the protruding inner tube during insertion of the guide wire in this instance.

A 10 French dilator is then placed over the wire and then removed followed by a 16 French dilator/peel being advanced over the wire. The dilator is then removed and a peritoneal dialysis catheter is then advance over the wire through the sheath. The wire is then removed as the catheter is placed into the peritoneum. A pocket is made into the rectus muscle for the deep cuff and a purse string is placed around the deep cuff once it has been positioned to reduce leaks especially for acute need catheters (<2 weeks). The catheter is then tunneled to the marked exit site either as a lateral or downward facing point to reduce risk of infection. The catheter is then re-checked for filling and draining to assure proper function prior to closing. Once this has been confirmed the catheter has a titanium connector and transfer set added. The incision is then closed with a dissolvable 3′0 moncryl suture.

Since various modifications can be made in my invention as herein above described, and many apparently widely different embodiments of same made, it is intended that all matter contained in the accompanying specification shall be interpreted as illustrative only and not in a limiting sense.

1. A Veress needle assembly for use with a peritoneal dialysis catheter guide wire, the assembly comprising:
a hollow needle having a hollow passage therein extending in a longitudinal direction between a first end of the hollow needle which is pointed and a second end of the hollow needle opposing the first end;
an inner tube received within the hollow passage of the hollow needle so as to be slidable relative to the hollow needle in the longitudinal direction, the inner tube being elongate in the longitudinal direction of the hollow needle between a first end of the inner tube which is in proximity to the first end of the hollow needle and a second end of the inner tube which is proximity to the second end of the hollow needle;
the inner tube being longitudinally slidable relative to the hollow needle between a first position in which the first end of the inner tube protrudes longitudinally beyond the pointed first end of the hollow needle and a second position in which the first end of the inner tube is retracted into the hollow needle relative to the first position so that the pointed first end of the hollow needle is unobstructed by the first end of the inner tube;
a biasing mechanism operatively coupled between the inner tube and the hollow needle at the second end of the hollow needle so as to bias the inner tube from the second position towards the first position;
a fluid coupler operatively connected to the second end of the inner tube for connection to a source of intravenous fluid;
the inner tube including a fluid passage extending longitudinally therethrough from the fluid coupler at the second end of the inlet tube to an outlet opening at the first end of the inner tube for passing the intravenous fluid therethrough;
the outlet opening of the fluid passage at the first end of the inner tube being oriented relative to the fluid passage so as to allow the peritoneal dialysis catheter guide wire to readily pass through the fluid passage and exit the outlet opening when inserted into the fluid passage through the second end of the inner tube.

2. The assembly according to claim 1 wherein the outlet opening is located in an end wall at the first end of the inner tube.

3. The assembly according to claim 1 wherein the outlet opening is concentric with the fluid passage in the inner tube.

4. The assembly according to claim 1 wherein the outlet opening is reduced in diameter relative to an outer diameter of the inner tube so as to define an annular rim portion about the outlet opening in an end wall of the inner tube at the first end thereof.

5. The assembly according to claim 4 wherein an outer surface of the annular rim portion is generally convex.

6. A method of guiding insertion of a peritoneal dialysis catheter guide wire, the method comprising:
providing a needle assembly comprising: (i) a hollow needle having a hollow passage therein extending in a longitudinal direction between a first end of the hollow needle which is pointed and a second end of the hollow needle opposing the first end, (ii) an inner tube received within the hollow passage of the hollow needle so as to be slidable relative to the hollow needle in the longitudinal direction in which the inner tube is elongate in the longitudinal direction of the hollow needle between a first end of the inner tube which is in proximity to the first end of the hollow needle and a second end of the inner tube which is proximity to the second end of the hollow needle, and in which the inner tube is longitudinally slidable relative to the hollow needle between a first position in which the first end of the inner tube protrudes longitudinally beyond the pointed first end of the hollow needle and a second position in which the first end of the inner tube is retracted into the hollow needle relative to the first position so that the pointed first end of the hollow needle is unobstructed by the first end of the inner tube, (iii) a biasing mechanism operatively coupled between the inner tube and the hollow needle at the second end of the hollow needle so as to bias the inner tube from the second position towards the first position, and (iv) a fluid passage extending longitudinally through the inner tube from the second end of the inlet tube to an outlet opening at the first end of the inner tube for passing the intravenous fluid therethrough;
and inserting the peritoneal dialysis catheter guide wire into the fluid passage of the inner tube through the second end of the inlet tube so that the guide wire exits the outlet opening of inner tube.
